SEQUENCE LISTING (5 sequences)

SEQ ID NO: 1 - DNA, 23 bp, Artificial Sequence
  Description of Artificial Sequence Synthetic oligonucleotide
ccggtcatttgttttgagaccgg

SEQ ID NO: 2 - DNA, 24 bp, Artificial Sequence
  Description of Artificial Sequence Synthetic oligonucleotide
acggtcatttgttttgagaccgtt

SEQ ID NO: 3 - DNA, 23 bp, Artificial Sequence
  Description of Artificial Sequence Synthetic oligonucleotide
ccggtcatttgttttgagaccgg

SEQ ID NO: 4 - DNA, 23 bp, Artificial Sequence
  Description of Artificial Sequence Synthetic oligonucleotide
ccggtcatttgttttgagaccgg

SEQ ID NO: 5 - DNA, 21 bp, Artificial Sequence
  Description of Artificial Sequence Synthetic oligonucleotide
aggtggcctttccaatacaat

(19) United States
(12) Patent Application Publication
(10) Pub. No.: US 2009/0197254 A1
(43) Pub. Date: Aug. 6, 2009

(54) VARIANT SCORPION PRIMERS FOR NUCLEIC ACID AMPLIFICATION AND DETECTION

(76) Inventor: Ming-Chou Lee, Mission Viejo, CA (US)

Correspondence Address: FOLEY & LARDNER LLP, P.O. BOX 80278, SAN DIEGO, CA, 92138-0278, US

(21) Appl. No.: 11/957,334

(22) Filed: Dec. 14, 2007

(51) Int. Cl. C12Q 1/68; C12P 19/34

(52) U.S. Cl. 435/6; 435/91.2

(57) ABSTRACT

Disclosed herein are methods of detecting target nucleic acids. In particular, methods for avoiding loss of the fluorescent label form an amplicon that is generated using a Scorpion primer and a polymerase with 5′ exonuclease activity. The methods use a Scorpion primer which comprises a fluorophore, a quencher, and in 5′ to 3′ order, a probe region, a linker region and a primer region, wherein the quencher is located at or near the 5′ end, and, wherein the primer is complementary to the target nucleic acid and the probe region hybridizes to a complementary sequence in an extension product of the primer. The methods provide for detection of target nucleic acids in simplex or multiplex formats.

### FIELD OF THE INVENTION

The present invention relates generally to the field of nucleic acid amplification and detection. In particular, the present invention relates to increasing signal output for primers and integrated probes during amplification of a target nucleic acid sequence.

### BACKGROUND OF THE INVENTION

The following description is provided to assist the understanding of the reader. None of the information provided or references cited is admitted to be prior art to the present invention.

Amplification of target nucleic acids continues to be important for molecular diagnostics and drug discovery. Nucleic acid assays for the detection and identification of diseases and pathogens depend upon reliable and efficient amplification of the target molecules. Various nucleic acid amplification techniques are known in the art. See e.g. Holland et al. (1991) PNAS 88: 7276, 7280; and U.S. Pat. No. 5,210,015. Amplification by polymerase chain reaction (PCR) is based on repeated cycles of the following steps: denaturation of double-stranded DNA followed by oligonucleotide primer annealing to the DNA template, and primer extension by a nucleic acid polymerase. The oligonucleotide primers used in PCR are designed to anneal to opposite strands of the DNA, and are positioned so that the nucleic acid polymerase-catalyzed extension product of one primer can serve as the template strand for the other primer. The PCR amplification process results in the exponential increase of discrete DNA fragments whose length is defined by the 5′ ends of the oligonucleotide primers. One of the major advance for PCR-based nucleic acid detection, quantification and genotyping has been the development of homogenous, closed-tube assays using fluorescence detection that facilitate high-throughput detection and minimize the likelihood of false-positive results owing to carryover contamination.

Nucleic acid amplification and detection may also be used to distinguish between closely related nucleotide sequences. In some instances, nucleotide sequences differ by only one or a few nucleotides, as in the case of many allelic sequences. For example, single nucleotide polymorphisms (SNPs) refer to alleles that differ by a single nucleotide. Even this single nucleotide difference can, at least in some instances, change the associated genetic response or traits. Allele-specific primers and probes can be used in nucleic acid amplification to discriminate between these sequences. Moreover, to determine which allele is present in a sample, nucleic acid amplification may be sensitive enough to distinguish between closely related sequences. Such methods can be a powerful tool for the identification of pathogens and disease.

A method for target nucleic acid detection has been described under the name “Scorpion.” A “Scorpion detection system” refers to a method for real-time PCR which utilizes a bi-functional molecule (referred to herein as a “Scorpion” or “Scorpion primer”), which contains a PCR primer element covalently linked by a polymerase-blocking group to a probe element. Additionally, each Scorpion contains a fluorophore that interacts with a quencher. See e.g. Whitcombe et al.: Detection of PCR products using self-probing amplicons and fluorescence. Nat. Biotechnol. 1999 August; 17(8): 804-7; Thelwell et al.: Mode of action and application of Scorpion primers to mutation detection. Nucleic Acids Res. 2000 Oct. 1; 28(19): 3752-61; U.S. Pat. No. 6,326,145; U.S. Pat. No. 6,365,729; US 2003 0087240 A1. During an amplification utilizing a Scorpion primer, the probe element of the Scorpion folds backwards on itself, similar looking to the tail of the scorpion animal, to hybridize to a complementary sequence in an extension product of the primer.

### SUMMARY OF THE INVENTION

The present invention relates to methods of detecting target nucleic acids in a sample. The methods of detection involve amplification of the target nucleic acid using a pair of primers that includes a variant Scorpion primer and a polymerase with endonuclease or 5′ exonuclease activity.

In one aspect, the present invention provides a method of avoiding loss of a fluorescent label from an amplicon generated by amplification of a target nucleic acid using a primer pair that includes a variant Scorpion primer and a polymerase with endonuclease or 5′ exonuclease activity. The method comprises amplifying a target nucleic acid with a pair of primers wherein one of the primers of the pair is a Scorpion primer comprising a fluorophore, a quencher, and in 5′ to 3′ order, a probe region, a linker region and a primer region, wherein the quencher is located at or near the 5′ end, and wherein the primer region is complementary to the target nucleic acid and the probe region hybridizes to a complementary sequence in an extension product of the primer region. In one embodiment, the Scorpion primer comprises in 5′ to 3′ order, a quencher, a probe region, a fluorophore, a linker region, and a primer region.

In one embodiment, the quencher and the fluorophore are found in spatial proximity to one another in the inactive form, and which are separated from one another by the hybridization of the probe to an amplification product. In one embodiment, the tailed primer may comprise a self-complementary stem duplex to place the quencher and fluorophore in close proximity under suitable hybridization conditions. For example, the self-complementary stem duplex may be formed by nucleotide sequences flanking the probe region of the tailed primer.

In one embodiment, the probe region of the tailed primer remains uncopied during amplification. This may be accomplished by placing a polymerase blocking moiety in the linker region between the probe region and the primer region. In a particular embodiment, the polymerase blocking moiety is a hexethylene glycol monomer.

In various embodiments, the quencher is any suitable non-fluorescent moiety, which absorbs the florescence from the fluorophore. For example, the quencher may have an excitation frequency near the emission frequency of the fluorophore, but does not emit the absorbed energy as light. In one embodiment, the quencher is a black hole quencher. In one embodiment, the fluorophore is FAM or ROX.

The methods of the invention may be used to detect a variety of target nucleic acids, e.g., target nucleic acids associated with a disease or pathogen. In one embodiment, the primer region, the probe region, or both the primer and probe regions are specific to particular alleles of the target nucleic acids.

### BRIEF DESCRIPTION OF THE DRAWINGS

FIG. 1 is a schematic representation of the generation of cleavage products from Scorpion primers. The binding of a primer element to a target nucleic acid is followed by hybridization of the probe element. FIG. 1A shows the configuration of a standard Scorpion primer and the cleavage of the fluorophore that would result by a nucleic acid polymerase having endonuclease or 5′ to 3′ exonuclease activity. FIG. 1B shows the configuration of a DQS Scorpion primer and the predicted cleavage of the quencher. It is believed that the enzyme may cleave the quencher from the Scorpion primer, however such fragments do not diminish the resulting fluorescent activity of the fluorophore associated with the amplicon.

FIG. 2 shows graphs of real-time amplification of a dilution series of PVL DNA using Taq polymerase with primers SFP2, SFP4, SFP5, and SFP6 (panels A-D, respectively).

FIG. 3 shows graphs of real-time amplification of a dilution series of PVL DNA using Pfu polymerase with primers SFP2, SFP4, SFP5, and SFP6 (panels A-D, respectively).

FIG. 4 shows the formation of detectable cleavage products from standard Scorpion primers using Taq polymerase, but not DQS Scorpion primers (SFP4) according to the present invention.

### DETAILED DESCRIPTION

Disclosed herein are methods of detecting target nucleic acids. In particular, methods for increasing the amount of fluorescently labeled amplicon associated with Scorpion technology are described. The methods provide for detection of target nucleic acids in simplex or multiplex formats for any purpose, e.g., gene copy number determination and SNP-genotyping.

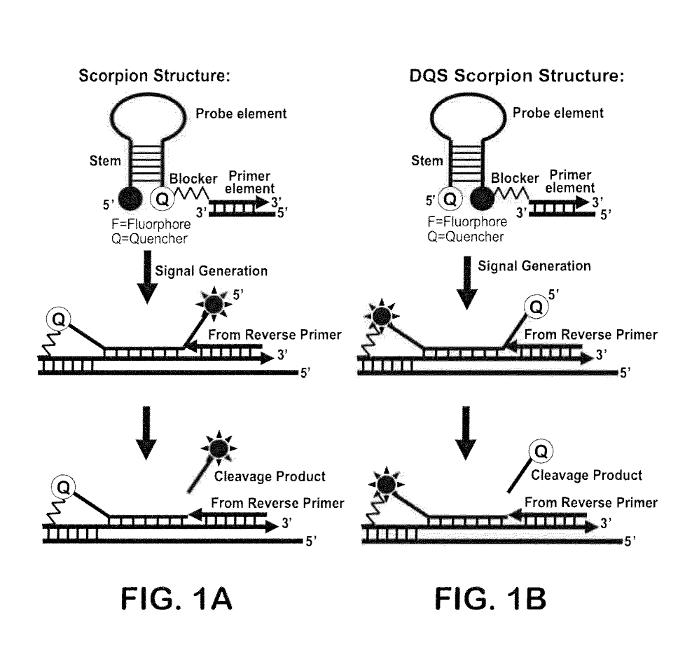

A method for target nucleic acid detection has been described under the name “Scorpion” (see, e.g. Whitcombe et al.: Detection of PCR products using self-probing amplicons and fluorescence. Nat Biotechnol. 1999 August; 17(8): 804-7; Thelwell et al.: Mode of action and application of Scorpion primers to mutation detection. Nucleic Acids Res. 2000 Oct. 1; 28(19): 3752-61; U.S. Pat. No. 6,326,145; U.S. Pat. No. 6,365,729; US 2003 0087240 A1). The present inventors discovered that there is a significant loss of fluorescent label from the amplicon generated from certain Scorpion primers by cleavage of the fluorophore from Scorpion when a nucleic acid polymerase having 5′ to 3′ exonuclease activity is used in the assay (See FIG. 1). Cleavage products are shown in FIG. 1A. These Scorpion primers which fail to retain the fluorophore are of the common type (referred to herein as “standard Scorpion primers” or just “Scorpion primers”) and comprise in 5′ to 3′ order, a fluorophore, a probe region, a quencher molecule, a linker region and a primer region. As is typical of Scorpions, the primer region is complementary to the target nucleic acid and the probe region hybridizes to a complementary sequence in an extension product of the primer region.

In response to this unexpected result, the present inventors discovered a Scorpion design that effectively retains the fluorophore on the amplicon during amplification with a nucleic acid polymerase having endonuclease or 5′ to 3′ exonuclease activity. This modified Scorpion primer, referred to as “DQS” (Dye-Quencher-Switched) comprise a fluorophore, a quencher, and in 5′ to 3′ order, a probe region, a linker region and a primer region, wherein the quencher is located at or near the 5′ end, and wherein the primer region is complementary to the target nucleic acid and the probe region hybridizes to a complementary sequence in an extension product of the primer region. In one embodiment, the DQS Scorpion comprises in 5′ to 3′ order, a quencher, a probe region, a fluorophore, a linker region, and a primer region. In this method, the fluorescent label from the DQS Scorpion primer is not cleaved from the amplicon by the polymerase. It may be that the enzyme cleaves the quencher from the amplicon as depicted in FIG. 1B, but this does not lead to loss of the fluorophore from the amplicon. The ability to retain the fluorophore in the amplicon assists, for example, in post amplification analysis of the amplicon where the fluorescent labeled is used to identify the amplicon. The additional quantities of labeled amplicon can provide for more post amplification assays, greater precision and/or sensitivity in such post amplification assays and the possibility of more accurate quantitative analysis reflective of the amplicon produced. Thus, the methods of the invention avoid the accumulation of non-fluorescently labeled amplicon resulting from amplification using standard Scorpion primers and a polymerase with endonuclease or 5′ exonuclease activity.

Units, prefixes, and symbols may be denoted in their accepted SI form. Unless otherwise indicated, nucleic acids are written left to right in 5′ to 3′ orientation; amino acid sequences are written left to right in amino to carboxy orientation. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes.

The terms “a” and “an” as used herein mean “one or more” unless the singular is expressly specified.

As used herein, “about” means plus or minus 10% unless otherwise indicated.

The terms “amplification” or “amplify” as used herein includes methods for copying a target nucleic acid, thereby increasing the number of copies of a selected nucleic acid sequence. Amplification may be exponential or linear. A target nucleic acid may be either DNA or RNA. The sequences amplified in this manner form an “amplicon” or “amplification product.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp 13-20; Wharam, et al., Nucleic Acids Res. 2001 Jun. 1; 29(11):E54-E54; Hafner, et al., Biotechniques 2001 30(4):852-6, 858, 860; Zhong, et al., Biotechniques 2001 30(4):852-6, 858, 860.

The term “complement” “complementary” or “complementarity” as used herein with reference to polynucleotides (i.e., a sequence of nucleotides such as an oligonucleotide or a target nucleic acid) refers to standard Watson/Crick pairing rules. The complement of a nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in natural nucleic acids may be included in the nucleic acids described herein; these include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be a sequence of RNA complementary to the DNA sequence or its complement sequence, and can also be a cDNA. The term “substantially complementary” as used herein means that two sequences specifically hybridize (defined below). The skilled artisan will understand that substantially complementary sequences need not hybridize along their entire length.

As used herein, the term “detecting” used in context of detecting a signal from a detectable label to indicate the presence of a target nucleic acid in the sample does not require the method to provide 100% sensitivity and/or 100% specificity. As is well known, “sensitivity” is the probability that a test is positive, given that the sample has a target nucleic acid sequence, while “specificity” is the probability that a test is negative, given that the sample does not have the target nucleic acid sequence. A sensitivity of at least 50% is preferred, although sensitivities of at least 60%, at least 70%, at least 80%, at least 90% and at least 99% are clearly more preferred. A specificity of at least 50% is preferred, although sensitivities of at least 60%, at least 70%, at least 80%, at least 90% and at least 99% are clearly more preferred. Detecting also encompasses assays with false positives and false negatives. False negative rates may be 1%, 5%, 10%, 15%, 20% or even higher. False positive rates may be 1%, 5%, 10%, 15%, 20% or even higher.

A “fragment” in the context of a nucleic acid refers to a sequence of contiguous nucleotide residues which are at least about 5 nucleotides, at least about 7 nucleotides, at least about 9 nucleotides, at least about 111 nucleotides, or at least about 17 nucleotides. The fragment is typically less than about 300 nucleotides, less than about 100 nucleotides, less than about 75 nucleotides, less than about 50 nucleotides, or less than 30 nucleotides. In certain embodiments, the fragments can be used in polymerase chain reaction (PCR) or various hybridization procedures to identify or amplify identical or related parts of mRNA or DNA molecules. A fragment or segment may uniquely identify each polynucleotide sequence of the present invention.

As used herein, “labels” are chemical or biochemical moieties useful for labeling a nucleic acid (including a single nucleotide), amino acid, or antibody. “Labels” include fluorescent agents, chemiluminescent agents, chromogenic agents, quenching agents, radionuclides, enzymes, substrates, cofactors, inhibitors, magnetic particles, and other moieties known in the art. “Labels” are capable of generating a measurable signal and may be covalently or noncovalently joined to an oligonucleotide or nucleotide (e.g., a non-natural nucleotide).

The term “multiplex PCR” as used herein refers to an assay that provides for simultaneous amplification of two or more products within the same reaction vessel. Each product is primed using a distinct primer pair. A multiplex reaction may further include labeled primers each product, that are detectably labeled with different detectable moieties.

As used herein, “nucleic acid,” “nucleotide sequence,” or “nucleic acid sequence” refer to a nucleotide, oligonucleotide, polynucleotide, or any fragment thereof and to naturally occurring or synthetic molecules. These phrases also refer to DNA or RNA of genomic or synthetic origin which may be single-stranded or double-stranded and may represent the sense or the antisense strand, or to any DNA-like or RNA-like material. An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. RNA may be used in the methods described herein and/or may be converted to cDNA by reverse-transcription for use in the methods described herein.

As used herein, the term “oligonucleotide” refers to a short polymer composed of deoxyribonucleotides, ribonucleotides or any combination thereof. Oligonucleotides are generally between about 10, 11, 12, 13, 14 or 15 to about 150 nucleotides (nt) in length, more preferably about 10, 11, 12, 13, 14, or 15 to about 70 nt, and most preferably between about 18 to about 26 nt in length. The single letter code for nucleotides is as described in the U.S. Patent Office Manual of Patent Examining Procedure, section 2422, table 1. In this regard, the nucleotide designation “R” means purine such as guanine or adenine, “Y” means pyrimidine such as cytosine or thymidine (uracil if RNA); and “M” means adenine or cytosine. An oligonucleotide may be used as a primer or as a probe.

As used herein, a “primer” for amplification is an oligonucleotide that is complementary to a target nucleotide sequence and leads to addition of nucleotides to the 3′ end of the primer in the presence of a DNA or RNA polymerase. The 3′ nucleotide of the primer should generally be identical to the target sequence at a corresponding nucleotide position for optimal expression and/or amplification. The term “primer” as used herein includes all forms of primers that may be synthesized including peptide nucleic acid primers, locked nucleic acid primers, phosphorothioate modified primers, labeled primers, and the like.

An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. Oligonucleotides used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

“Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Equations for calculating Tₘ and conditions for nucleic acid hybridization are known in the art.

As used herein, a primer is “specific” for a nucleic acid if the oligonucleotide has at least 50% sequence identity with a portion of the nucleic acid when the oligonucleotide and the nucleic acid are aligned. A primer that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity. Sequence identity can be determined using a commercially available computer program with a default setting that employs algorithms well known in the art (e.g., BLAST). As used herein. Sequences that have “high sequence identity” have identical nucleotides at least at about 50% of aligned nucleotide positions, preferably at least at about 60% of aligned nucleotide positions, and more preferably at least at about 75% of aligned nucleotide positions.

As used herein, the term “sample” or “biological sample” may comprise clinical samples, isolated nucleic acids, or isolated microorganisms. In preferred embodiments, a sample is obtained from a biological source (i.e., a “biological sample”), such as tissue, bodily fluid, or microorganisms collected from a subject. Sample sources include, but are not limited to, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). The term “patient sample” as used herein refers to a sample obtained from a human seeking diagnosis and/or treatment of a disease.

The terms “target nucleic acid” or “target sequence” as used herein refer to a sequence which includes a segment of nucleotides of interest to be amplified and detected. Copies of the target sequence which are generated during the amplification reaction are referred to as amplification products, amplimers, or amplicons. Target nucleic acid may be composed of segments of a chromosome, a complete gene with or without intergenic sequence, segments or portions of a gene with or without intergenic sequence, or sequence of nucleic acids which probes or primers are designed. Target nucleic acids may include a wild-type sequence(s), a mutation, deletion or duplication, tandem repeat regions, a gene of interest, a region of a gene of interest or any upstream or downstream region thereof. Target nucleic acids may represent alternative sequences or alleles of a particular gene. Target nucleic acids may be derived from genomic DNA, cDNA, or RNA. As used herein target nucleic acid may be DNA or RNA extracted from a cell or a nucleic acid copied or amplified therefrom.

As used herein, the term “Scorpion detection system” refers to a method for real-time PCR. This method utilizes a bi-functional molecule (referred to herein as a “Scorpion” or “Scorpion primer”), which contains a PCR primer element covalently linked by a polymerase-blocking group to a probe element. Additionally, each Scorpion primer contains a fluorophore that interacts with a quencher. The typical or standard Scorpion primer is depicted in FIG. 1. In use the probe region of the Scorpion hybridizes to a complementary sequence in an extension product of the primer corresponding to the target nucleic acid.

Scorpion primers that are useful in the methods of the invention are referred to herein as “DQS” Scorpion primers (“Dye-Quencher-Switched” Scorpion primers). DQS Scorpions comprise a fluorophore, a quencher, and in 5′ to 3′ order, a probe region, a linker region and a primer region, wherein the quencher is located at or near the 5′ end, and wherein the primer region is complementary to the target nucleic acid and the probe region hybridizes to a complementary sequence in an extension product of the primer region. In suitable embodiments, the primer region and the probe region of the DQS Scorpion are arranged such that the probe region remains single stranded in the PCR amplification products. Typically, a blocking moiety is sited between the primer region of the and the probe region of the DQS Scorpion. The blocking moiety prevents polymerase mediated copying of the tail region of the primer template.

The probe region of the DQS Scorpion comprises a sequence which may hybridize to a complementary target sequence in the primer extension product. The spacing on a DNA strand between the probe region and its complementary sequence within the amplicon may be as little as 30 bases (that is directly abutting the primer region) or may be as much as about 200-300 bases. The efficiency of the unimolecular interaction is expected to decline as this distance increases. In some embodiments, the spacing is less than 200 base pairs, less than 100 base pairs, less than 50 base pairs, less than 40 base pairs, less than 30 base pairs, less than 25, less than 20 base pairs, less than 15, 10 or even 5 base pairs from the primer region.

The DQS Scorpions used in the methods of the invention further comprise a quencher and a fluorophore, wherein the quencher is located further 5′ in the molecule relative to the fluorophore. Hybridization of the probe region in the tail of the bifunctional oligonucleotide to a complementary sequence in the primer extension product corresponding to the target nucleic acid causes a detectable change in the signaling system. In one embodiment, the signaling system is a two-component system where a signal is created or reduced and/or abolished when the two components are brought into close proximity with one another. Alternatively a signal is created or reduced and/or abolished when the two components are separated following binding of the target binding region.

The methods of the invention are applicable in different embodiments. In one embodiment, the DQS Scorpion is used as an amplification primer in an amplification system such as the polymerase chain reaction (PCR). Prior to amplification, the probe region exists in a quenched configuration where the fluorophore and the quencher are kept in close proximity. After initial denaturation, annealing and extension, the amplicon comprises a region complementary to the probe region at its 5′ end. Upon a second round of denaturation and annealing, the probe region hybridizes to the newly synthesized amplification product with great efficiency (a unimolecular interaction) and fluorescence remains unquenched. Unextended primers, however, will continue to exist in their quenched conformation.

Meanwhile, a “reverse” primer will have hybridized to this same strand and will be extended by a polymerase. It is believed that the tail of the DQS Scorpion, which hybridizes to a complementary sequence in the amplicon, may be cleaved by a polymerase having 5′ to 3′ exonuclease activity or endonuclease activity, thereby releasing the quencher moiety. Endonuclease activity refers to the cutting or nicking of a DNA at sites within the DNA molecule. By contrast, exonuclease activity refers to the cleavage of bonds, preferably phosphodiester bonds, between nucleotides one at a time from the end of a DNA molecule. Because the quencher is at the 5′ end of the DQS Scorpion, there is no loss of fluorescence from the amplicon.

The Scorpion primers used in the methods of the invention may be used in place of conventional amplification primers, such as PCR primers. The probe region is not expected to interfere with the amplification function. In one embodiment, multiple primer/probe molecules may be used in an allele-specific assay (e.g. detecting wild-type and mutant alleles). Each allele-specific primer may be labeled with different fluorophores, thus permitting single tube genotyping—that is, both reactions are run in the same tube and the amplicons are distinguished by their characteristic signal. Alternatively, the signaling entity may carry the allelic specificity: the primers are standard (non-allele specific) primers and two different probe regions matching the two allelic variants are introduced on two variants of one of the primers. Discrimination between the alleles is achieved either by fluorescence wavelength or alternatively by the use of probe elements having the same fluorophore but different melting temperatures which may then be discerned by measuring the fluorescence over a temperature range.

It will be appreciated that the overall length of the primer region and/or probe region will be determined principally by the intended functions of its individual components. In general, the primer will be of at least 10 base pairs, such as at least 20, 30, 40 or 50 base pairs, for example 10-30 or 15-25 base pairs. The probe region of the bifunctional oligonucleotide hybridizes to the target nucleic acid, if present in the sample. The probe may be designed according to various practical considerations, i.e., amplicon size, annealing temperature, hairpin formation, etc. Target binding can be effected at any desired stringency, that is to say under appropriate hybridization stringency conditions the template binding region of the probe may hybridize to the template region (if present in the template) to the exclusion of other regions.

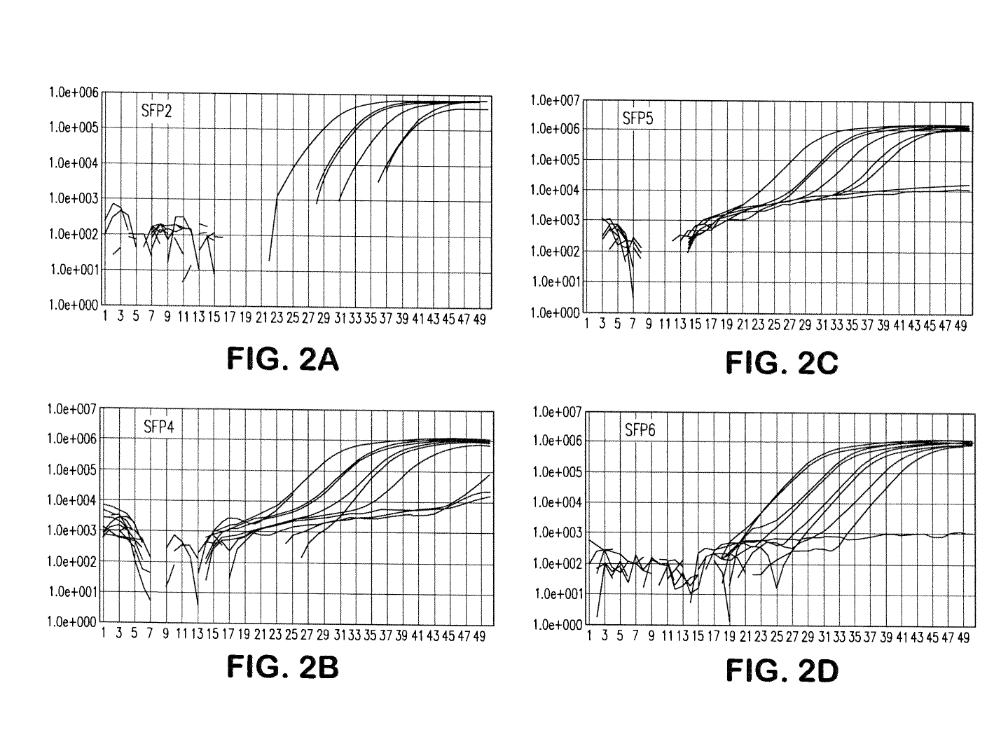

Probe regions are typically about 10-20 bases, about 15-25 bases, about 20-30 bases, or about 25-50 bases. Although depending upon the temperature at which measurements are to be taken, shorter (as little as 6 to 10 bases) probe regions may be used. In one embodiment, the bifunctional oligonucleotide comprises self complementary stems (also DNA, RNA, 2′-O-methyl RNA, PNA and their variants) which flank the probe region, such that hairpin formation by the two stems brings the Q/F pair together causing the fluorescence to be substantially quenched (FIG. 2). At higher temperatures, the stem duplex is disrupted and the fluorophore is unquenched; at lower temperatures, however, the stem duplex forms and the fluorescence is substantially off.

In certain embodiments, the DQS Scorpion primer may further comprise a linker. The linker separates the primer region and probe region. Optimum characteristics for the linker may be determined by routine experimentation. The linker may comprise less than 200 nucleotides, less than 100 nucleotides, less than 50 nucleotides, or less than 20 nucleotides. In suitable embodiments, the linker is less than about 50 nucleotides, so that the probe region is kept close to the complementary sequence in the target.

The linker may comprise a polymerase blocking moiety, which prevents polymerase mediated chain extension on the primer template. The polymerase blocking moiety prevents a read-through of the polymerase during primer extension. In one embodiment, the probe region is arranged such that the probe region remains single stranded after primer extension from the opposite primer. Thus, the tail region is non-amplifiable in the PCR amplification products. In some embodiments, the polymerase blocking moiety is a deoxyribose chain that lacks the bases (i.e. a chain of abasic sites) or a string of modified nucleotides that allows hybridization but does not allow DNA polymerase synthesis, for example iso-guanine nucleotide or iso-cytosine nucleotides. In one embodiment, the polymerase blocking moiety is hexethylene glycol (HEG) monomer. In another embodiment, the linker comprises material such as 2-O-alkyl RNA which will not permit polymerase mediated replication of a complementary strand.

The DQS Scorpions described herein may comprise one or more labels, such as a fluorophore and/or a quencher. Nucleotides and oligonucleotides can be labeled by incorporating moieties detectable by spectroscopic, photochemical, biochemical, immunochemical, or chemical assays. The method of linking or conjugating the label to the nucleotide or oligonucleotide depends on the type of label(s) used and the position of the label on the nucleotide or oligonucleotide.

In suitable embodiments, the DQS Scorpions used herein bear a fluorophore and a quencher, by means of which a detection can be made of whether a hybridization has occurred. Various signal systems are known to the person skilled in the art for this purpose. Thus, among other things, fluorescent dye/quencher pairs, intercalating dyes and dye pairs, which produce signals via fluorescence-resonance energy transfer (FRET) can be used.

In some embodiments, two interactive labels may be used on a single oligonucleotide with due consideration given for maintaining an appropriate spacing of the labels on the oligonucleotide to permit the separation of the labels during oligonucleotide hydrolysis. Consideration is given to having an appropriate spacing of the labels between the oligonucleotides when hybridized.

The DQS Scorpions of the disclosed methods may be labeled with a “fluorescent dye” or a “fluorophore.” As used herein, a “fluorescent dye” or a “fluorophore” is a chemical group that can be excited by light to emit fluorescence. Some suitable fluorophores may be excited by light to emit phosphorescence. Dyes may include acceptor dyes that are capable of quenching a fluorescent signal from a fluorescent donor dye. Dyes that may be used in the disclosed methods include, but are not limited to, the following dyes and/or dyes sold under the following trade names: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-HAT (Hydroxy Tryptamine); 5-Hydroxy Tryptamine (HAT); 5-ROX (carboxy-X-rhodamine); 6-Carboxyrhodamine 6G; 6-JOE; 6-carboxyfluorescein (6-FAM); 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy FI-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue Calcium Crimson™; Calcium Green; Calcium Orange; Calcofluor White; Cascade Blue™; Cascade Yellow; Catecholamine; CCF₂ (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiICi18(5)); DIDS; Dihydrorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF₂); GFP(S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium lodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF I; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 1; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TE™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The DQS Scorpion used herein may be labeled with a donor fluorophore and an acceptor fluorophore (or quencher dye) that are present in the oligonucleotides at positions that are suitable to permit FRET (or quenching). In some embodiments, the Scorpion primers may be labeled with a quencher. Interactive labels may utilize proximal quenching or FRET quenching. In proximal quenching (a.k.a. “contact” or “collisional” quenching), the donor is in close proximity to the quencher moiety such that energy of the donor is transferred to the quencher, which dissipates the energy as heat as opposed to a fluorescence emission. In FRET quenching, the donor fluorophore transfers its energy to a quencher which releases the energy as fluorescence at a higher wavelength. Proximal quenching requires very close positioning of the donor and quencher moiety, while FRET quenching, also distance related, occurs over a greater distance (generally 1-10 nm, the energy transfer depending on R-6, where R is the distance between the donor and the acceptor). Thus, when FRET quenching is involved, the quenching moiety is an acceptor fluorophore that has an excitation frequency spectrum that overlaps with the donor emission frequency spectrum. When quenching by FRET is employed, the assay may detect an increase in donor fluorophore fluorescence resulting from increased distance between the donor and the quencher (acceptor fluorophore) or a decrease in acceptor fluorophore emission resulting from decreased distance between the donor and the quencher (acceptor fluorophore).

Suitable quenchers include Dabcyl, Iowa Black™, or black hole quenchers sold under the trade name “BHQ” (e.g., BHQ-0, BHQ-1, BHQ-2, and BHQ-3, Biosearch Technologies, Novato, Calif.). Dark quenchers also may include quenchers sold under the trade name “QXL™” (Anaspec, San Jose, Calif.). Dark quenchers also may include DNP-type non-fluorophores that include a 2,4-dinitrophenyl group.

The labels can be attached to the oligonucleotides directly or indirectly by a variety of techniques. Using commercially available phosphoramidite reagents, one can produce oligonucleotides containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite, and can label them using protocols described in, for example, PCR Protocols: A Guide to Methods and Applications, ed. by Innis et al., Academic Press, Inc., 1990. Methods for incorporating oligonucleotide functionalizing reagents having one or more sulfhydryl, amino or hydroxyl moieties into the oligonucleotide reporter sequence, typically at the 5′ terminus, are described in U.S. Pat. No. 4,914,210, incorporated herein by reference. Labels at the 3′ terminus, for example, can employ polynucleotide terminal transferase to add the desired moiety, such as for example, cordycepin, ³⁵S-dATP, and biotinylated dUTP.

In one embodiment, the interactive signal generating pair comprises a fluorophore and a quencher that can quench the fluorescent emission of the fluorophore. For example, a quencher may include a BHQ and the fluorophore may be FAM or ROX. Other fluorophore-quencher pairs have been described in Morrison, Detection of Energy Transfer and Fluorescence Quenching in No isotopic Probing, Blotting and Sequencing, Academic Press, 1995.

In one embodiment, the nucleic acid amplification is performed in a real-time homogeneous assay. A real-time assay is one that produces data indicative of the presence or quantity of a target molecule during the amplification process, as opposed to the end of the amplification process. A homogeneous assay is one in which the amplification and detection reagents are mixed together and simultaneously contacted with a sample, which may contain a target nucleic acid molecule. Thus, the ability to detect and quantify DNA targets in real-time homogeneous systems as amplification proceeds is centered in single-tube assays in which the processes required for target molecule amplification and detection take place in a single “closed-tube” reaction format.

Homogenous PCR methods (closed tube methods) offer the advantage that they do not require the operator to perform manual separation of the amplified target by means of gel electrophoresis or other methods. Once setup is complete, target detection can be accomplished without additional manipulation of the sample. Such assays facilitate high throughput by monitoring the accumulation of fluorescence in a closed tube. Once the sample extract and reagents are combined, the tube is sealed and does not need to be opened again. This method minimizes the likelihood of false-positive results due to carryover contamination of the sample, facilitates sample tracking, and significantly reduces hands-on processing time.

The template nucleic acid is any convenient nucleic acid for analysis. This DNA target may have been derived from a reverse transcription (RT) reaction. Indeed, the primer of the invention may be used in the RT reaction itself and be used directly, without further amplification. Other in vitro amplification techniques such as ligase chain reaction (LCR), OLA, NASBA and Strand Displacement Amplification (SDA) may also be suitable. It is important however that there is a single stranded intermediate which allows the target binding region to hybridize to a complementary sequence in the primer extension product.

Sources of sample nucleic acid include human cells such circulating blood, buccal epithelial cells, cultured cells and tumor cells. Also other mammalian tissue, blood and cultured cells are suitable sources of template nucleic acids. In addition, viruses, bacteriophage, bacteria, fungi and other micro-organisms can be the source of nucleic acid for analysis. The DNA may be genomic or it may be cloned in plasmids, bacteriophage, bacterial artificial chromosomes (BACs), yeast artificial chromosomes (YACs) or other vectors. RNA may be isolated directly from the relevant cells or it may be produced by in vitro priming from a suitable RNA promoter or by in vitro transcription. Samples of nucleic acids may be prepared according to various methods (See e.g., Sambrook et al., Molecular Cloning. A Laboratory Manual, 2nd edition, Cold Spring Harbor Press, Cold Spring Harbor, N.Y. (1989)).

The present invention may be used for the detection of variation in genomic DNA whether human, animal or other. It finds particular use in the analysis of inherited or acquired diseases or disorders. In addition to the gene based diagnostics of human heritable disease, the invention will be useful in the detection of amplicons from other sources. A particular use is in the detection of infectious agents (bacteria, viruses etc), such as HIV, where the combination of allele specific priming and allelic discrimination via the target binding region offers opportunities to monitor the emergence of particular variants of HIV within a virus population in a patient. Other infectious agents for which quantitative data (measured by real time PCR) would be helpful include Hepatitis C virus. In other medical microbiology applications it is important to be able to detect and quantify particular species of microorganism.

In various embodiments, a polymerase enzyme is used in the amplification of nucleic acids. Suitable nucleic acid polymerases include, for example, polymerases capable of extending an oligonucleotide by incorporating nucleic acids complementary to a template oligonucleotide. For example, the polymerase can be a DNA polymerase. Enzymes having polymerase activity catalyze the formation of a bond between the 3′ hydroxyl group at the growing end of a nucleic acid primer and the 5′ phosphate group of a nucleotide triphosphate. These nucleotide triphosphates are usually selected from deoxyadenosine triphosphate (A), deoxythymidine triphosphate (T), deoxycytosine triphosphate (C) and deoxyguanosine triphosphate (G).

Because the relatively high temperatures necessary for strand denaturation during methods such as PCR can result in the irreversible inactivation of many nucleic acid polymerases, nucleic acid polymerase enzymes useful for performing the methods disclosed herein preferably retain sufficient polymerase activity to complete the reaction when subjected to the temperature extremes of methods such as PCR. Typically, the nucleic acid polymerase enzymes useful for the methods disclosed herein are thermostable nucleic acid polymerases. Suitable thermostable nucleic acid polymerases include, but are not limited to, enzymes derived from thermophilic organisms. Examples of thermophilic organisms from which suitable thermostable nucleic acid polymerase can be derived include, but are not limited to, Thermus aquaticus, Thermus thermophilus, Thermus flavus, Thermotoga neapolitana and species of the Bacillus, Thermococcus, Sulfobus, and Pyrococcus genera. Nucleic acid polymerases can be purified directly from these thermophilic organisms. However, substantial increases in the yield of nucleic acid polymerase can be obtained by first cloning the gene encoding the enzyme in a multicopy expression vector by recombinant DNA technology methods, inserting the vector into a host cell strain capable of expressing the enzyme, culturing the vector-containing host cells, then extracting the nucleic acid polymerase from a host cell strain which has expressed the enzyme. Suitable thermostable nucleic acid polymerases, such as those described above, are commercially available.

In addition, it will be recognized that RNA can be used as a sample and that a reverse transcriptase can be used to transcribe the RNA to cDNA. The transcription can occur prior to or during PCR amplification. Examples of reverse transcriptases that can be used include, but are not limited to, ImProm-II Reverse Transcriptase (Promega, Madison, Wis.), SuperScript III reverse transcriptase (Invitrogen, Calsbad, Calif.) and BD Powerscript Reverse Transcriptase (BD Biosciences, Franklin Lakes, N.J.). Methods for using reverse transcriptases to prepare and obtain cDNA molecules are well known in the art and are described in Sambrook, J. et al., Molecular Cloning: A Laboratory Manual, 2nd edition, Cold Spring Harbor Press, Cold Spring Harbor, N.Y. (1989).

In a suitable embodiment, real time PCR is performed using any suitable instrument capable of detecting fluorescence from one or more fluorescent labels. For example, real time detection on the instrument (e.g. a ABI Prism® 7900HT Sequence Detector) monitors fluorescence and calculates the measure of reporter signal, or Rn value, during each PCR cycle. The threshold cycle, or Ct value, is the cycle at which fluorescence intersects the threshold value. The threshold value is determined by the sequence detection system software or manually.

Using appropriate signaling systems (for example different fluorophores) it is possible to combine (multiplex) the output of several detectable primers/probes in a single reaction. The number of primers that may be used is limited only by experimental considerations.

In some embodiments, melting curve analysis may be used to detect an amplification product. Melting curve analysis involves determining the melting temperature of an nucleic acid amplicon by exposing the amplicon to a temperature gradient and observing a detectable signal from a fluorophore. Melting curve analysis is based on the fact that a nucleic acid sequence melts at a characteristic temperature called the melting temperature (Tₘ), which is defined as the temperature at which half of the DNA duplexes have separated into single strands. The melting temperature of a DNA depends primarily upon its nucleotide composition. Thus, DNA molecules rich in G and C nucleotides have a higher Tₘ than those having an abundance of A and T nucleotides.

Where a fluorescent dye is used to determine the melting temperature of a nucleic acid in the method, the fluorescent dye may emit a signal that can be distinguished from a signal emitted by any other of the different fluorescent dyes that are used to label the oligonucleotides. In some embodiments, the fluorescent dye for determining the melting temperature of a nucleic acid may be excited by different wavelength energy than any other of the different fluorescent dyes that are used to label the oligonucleotides. In some embodiments, the second fluorescent dye for determining the melting temperature of the detected nucleic acid is an intercalating agent. Suitable intercalating agents may include, but are not limited to SYBR™ Green 1 dye, SYBR™ dyes, Pico Green, SYTO dyes, SYTOX dyes, ethidium bromide, ethidium homodimer-1, ethidium homodimer-2, ethidium derivatives, acridine, acridine orange, acridine derivatives, ethidium-acridine heterodimer, ethidium monoazide, propidium iodide, cyanine monomers, 7-aminoactinomycin D, YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, and mixture thereof. In suitable embodiments, the selected intercalating agent is SYBR™ Green 1 dye.

By detecting the temperature at which the fluorescence signal is lost, the melting temperature can be determined. In the disclosed methods, each of the amplified target nucleic acids may have different melting temperatures. For example, each of these amplified target nucleic acids may have a melting temperature that differs by at least about 1° C., more preferably by at least about 2° C., or even more preferably by at least about 4° C. from the melting temperature of any of the other amplified target nucleic acids. By observing differences in the melting temperature(s) of the respective amplification products, one can confirm the presence or absence of the target nucleic acids in the sample.

To minimize the potential for cross contamination, reagent and master mix preparation, specimen processing and PCR setup, and amplification and detection are all carried out in physically separated areas.

### EXAMPLES

The present invention is further illustrated by the following examples, which should not be construed as limiting in any way.

### Example 1

### Cleavage of Scorpion Primers with Native Tag Polymerase

The experiment described in this example tested variant Scorpions, including the DQS Scorpion, in both 4× Pfu and native Taq chemistries and compared them to standard Scorpion primers. Four different primer/probes were designed to detect the Panton-Valentine Leukocidin (PVL) gene from Staphylococcus aureus. SFP2 is a standard Scorpion; SFP4 is a DQS Scorpion; SFP5 is a Scoprion having two HEG moieties flanking the probe region; and SFP 6 is a Scorpion having the first four nucleotides attached with a 2′-OMe group. The arrangement and nucleotide sequence of the oligonucleotides are shown in Table 1.

TABLE 1
Sequences of Standard and Variant Scorpion Oligonucleotides

| | | |
|---|---|---|
| Primer Name | Sequence | SEQ ID NO: |

| | | |
|---|---|---|
| SA2-SFP2 | 5′ (6-FAM)-CCGGTCATTTGTTTTGAGACCGG- | SEQ ID NO:1 |
| | (BHQ1)-(HEG)-AGGTGGCCTTTCCAATACAAT 3′ | |
| SA2-SFP4 | 5′ BHQ1-ACGGTCATTTGTTTTGAGACCGT-(T-6- | SEQ ID NO:2 |
| | FAM)-(HEG)-AGGTGGCCTTTCCAATACAAT 3′ | |
| SA2-SFP5 | 5′(6-FAM)-(HEG)-CCGGTCATTTGTTTTGAGA | SEQ ID NO:3 |
| | CCGG-(BHQ1)-(HEG)-AGGTGGCCTTTCCAAT | |
| | ACAAT 3′ | |
| SA2-SFP6 | 5′ (6-FAM)-(2′-MeO)C-(2′-MeO)C-(2′-MeO)G-(2′- | SEQ ID NO:4 |
| | MeO)G-TCATTTGTTTTGAGACCGG-(BHQ1)- | |
| | (HEG)-AGGTGGCCTTTCCAATACAAT 3′ | |

The reaction was conducted on an ABI 7500 Sequence Detection System using the following cycling conditions: 95° C. for 5 min; and 50 cycles of 95° C. for 10 sec and 50° C. for 35 sec.

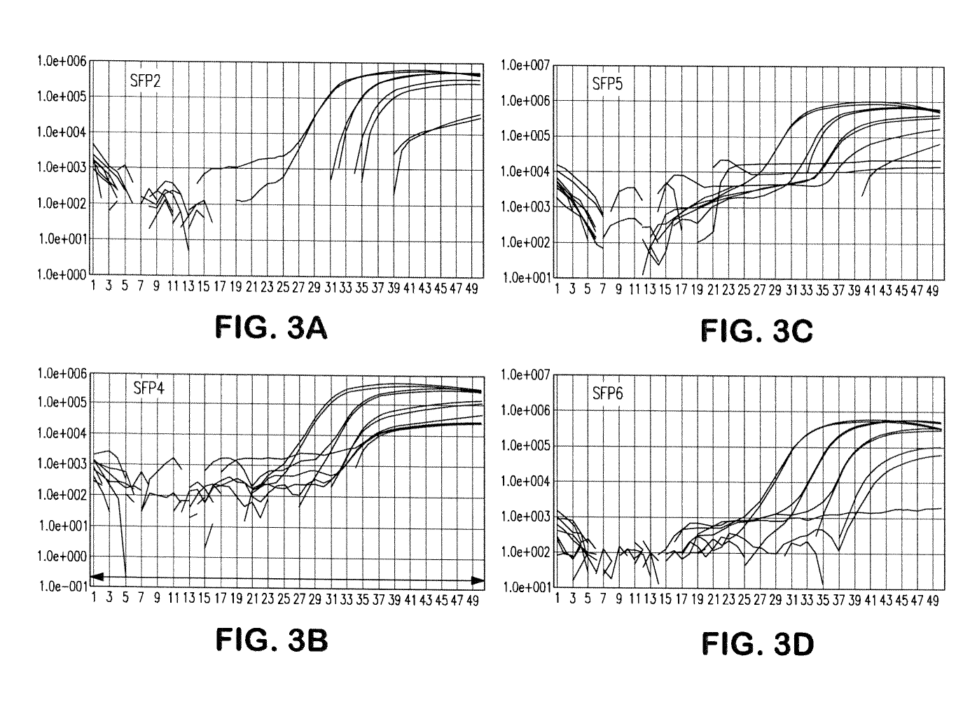

The results are shown in FIGS. 2 and 3, which depict real-time amplification plots using Taq polymerase or Pfu polymerase, respectively. Template-dependent amplification was seen with all PVL probe variants SFP2, SFP4, SFP5, and SFP6. As can be seen in FIG. 2, SFP2 (standard Scorpion, FIG. 2A) has steeper curve than SFP4 (DQS, FIG. 2B) indicating there were multiple events that contributed to the signal generation. Typically, signal generation in Scorpion assays occurs the moment the probe portion of the Scorpion hybridizes to the complementary sequence of the extended product. In reactions using the standard Scorpion (SFP2) or variants having a 5′ fluorophore (SFP5 and SFP6), the signal from cleavage of the fluorophore further contributed to the total signal, while this did not occur in the DQS Scorpion (SFP4).

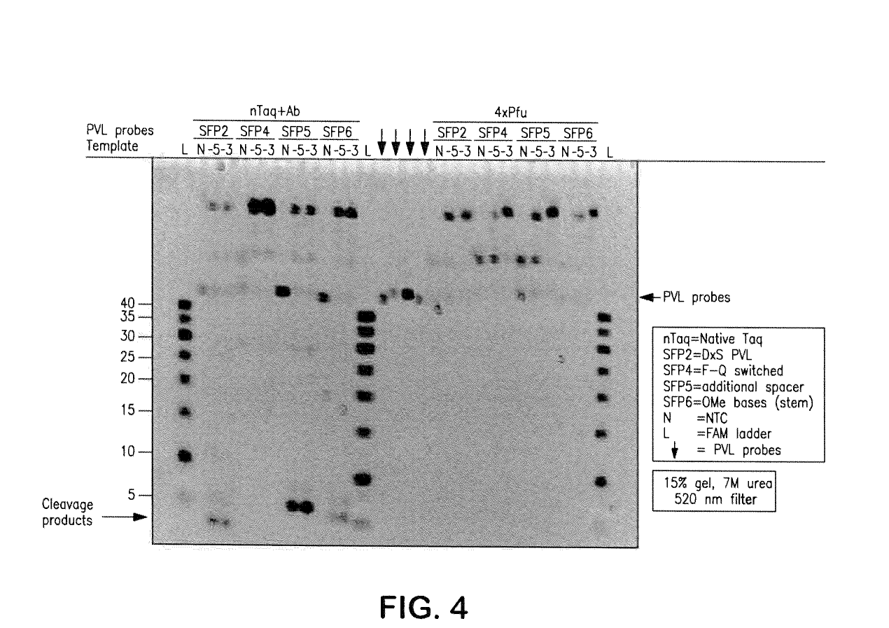

The amplification products were fractionated using a 15% gel containing 7M urea and detected using a scanner with a 520 nm filter. The results are shown in FIG. 4. No small fragments were observed from amplification with any of the Scorpion primers using Pfu polymerase. Template-dependent small fragments were detected with native Taq for SFP2, SFP5, and SFP6 primers, but not for SFP4, which has the quencher on the 5′ end of the Scorpion. Therefore, switching the position of the fluorophore and the quencher in the DQS Scorpion abolished the detection of small, FAM-labeled fragments.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

What is claimed is:

1. A method of avoiding loss of a fluorescent label from an amplicon generated by amplification of a target nucleic acid using a primer pair that includes a Scorpion primer and a polymerase with endonuclease or 5′ exonuclease activity, comprising amplifying a target nucleic acid with a pair of primers wherein one of the primers of the pair is a Scorpion primer comprising, a fluorophore, a quencher, and in 5′ to 3′ order, a probe region, a linker region and a primer region, wherein the quencher is located at or near the 5′ end, and wherein the primer region is complementary to the target nucleic acid and the probe region hybridizes to a complementary sequence in an extension product of the primer region.

2. The method of claim 1, wherein the Scorpion primer comprises in 5′ to 3′ order, a quencher, a probe region, a fluorophore, a linker region, and a primer region.

3. The method of claim 1, wherein Scorpion primer comprises a self-complementary stem duplex to place the quencher and fluorophore in spatial proximity under suitable hybridization conditions.

4. The method of claim 3, wherein the self-complementary stem duplex is formed by nucleotide sequences flanking the probe region of the tailed primer.

5. The method of claim 1, wherein the probe region of the Scorpion primer remains uncopied during amplification.

6. The method of claim 1, wherein the linker region comprises a polymerase blocking moiety.

7. The method of claim 6, wherein the polymerase blocking moiety is hexethylene glycol monomer.

8. The method of claim 1, wherein the fluorophore is selected from the group consisting of: Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5, 5-FAM, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

9. The method of claim 1, wherein the quencher is selected for the group consisting of: black hole quencher and Dabcyl.

10. The method of claim 1, wherein the sample is contacted in a multiplex reaction with one or more additional primer pairs, wherein one of the primers of each pair is a Scorpion primer comprising a fluorophore, a quencher, and in 5′ to 3′ order, a probe region, a linker region and a primer region, wherein the quencher is located at or near the 5′ end, and wherein the primer and probe regions are suitable for the amplification and detection of additional target nucleic acids.

11. The method of claim 10, wherein the fluorophores of each bifunctional oligonucleotide are different.

12. The method of claim 1, wherein hybridization of the primer region, the probe region, or both the primer and probe regions to the target nucleic acid is allele specific.

13. The method of claim 1, wherein the polymerase is a Taq polymerase.

14. The method of claim 1, wherein the amplification products are detected in a real-time PCR reaction.
